(19) United States
(12) Patent Application Publication
(10) Pub. No.: US 2016/0022843 A1
(43) Pub. Date: Jan. 28, 2016

(54) COATED MAGNETIC NANOPARTICLES FOR IMAGING ENHANCEMENT AND DRUG DELIVERY

(71) Applicant: EMORY UNIVERSITY, Atlanta, GA (US)

(72) Inventors: Hui MAO, Johns Creek, GA (US); Lilly YANG, Atlanta, GA (US); Jing HUANG, Decatur, GA (US)

(21) Appl. No.: 14/775,840

(22) PCT Filed: Mar. 17, 2014
(86) PCT No.: PCT/US2014/030365

§ 371 Date: Sep. 14, 2015

Related U.S. Application Data
(60) Provisional application No. 61/789,960, filed on Mar. 15, 2013.

(51) Int. Cl. A61K 49/18; A61K 47/48; A61K 41/00; A61K 49/12

(52) U.S. Cl. CPC A61K 49/1824; A61K 49/12; A61K 47/48884; A61K 47/48861; A61K 47/48246; A61K 41/00

(57) ABSTRACT

This disclosure is in the area of diagnostics and nanotechnology. This disclosure provides a method for synthesizing casein coated iron oxide nanoparticles to be used as an MRI contrasting agent and targeted drug delivery.

### CROSS-REFERENCE TO RELATED APPLICATIONS

This application is a 371 U.S.C of International Application PCT/US2014/030365, filed Mar. 17, 2014, which claims the benefit of priority to U.S. Provisional Application No. 61/789,960, filed Mar. 15, 2013, which applications are incorporated by reference in their entireties.

### STATEMENT REGARDING FEDERALLY FUNDED RESEARCH

This invention was made with government support under grants R01 CA154846-02, U01 CA151810-03, and P50CA128301 awarded by the National Institutes of Health. The Government has certain rights in the invention.

### FIELD

This disclosure is in the area of diagnostics and nanotechnology. In certain embodiments, it provides a method for synthesizing casein coated iron oxide nanoparticles to be used as a MRI contrasting agent and for targeted drug delivery.

### BACKGROUND

Magnetic iron oxide nanoparticles such as Fe₃0₄ and γ-Fe₂0₃ have recently attracted extensive attention in biomaterial development due to their applications in cell labeling, cell sorting, magnetic resonance imaging (MRI), and imaging-guided drug delivery. Chen et al. Triblock Copolymer Coated Iron Oxide Nanoparticle Conjugate for Tumor Integrin Targeting. See Biomaterials 2009, 30, 6912-6919, Laurent et al. Magnetic Iron Oxide Nanoparticles for Biomedical Applications. See Future Medicinal Chemistry 2010, 2, 427-449 and Huang et al. Improving the Magnetic Resonance Imaging Contrast and Detection Methods with Engineered Magnetic Nanoparticles. See Theranostics 2012, 2, 86-102.

Iron oxide nanoparticles synthesized via pyrolysis of organometallic iron precursors possess high uniformity and excellent magnetic properties but poor water solubility in biomedical applications, due to the inevitable hydrophobic residue, oleic acid, on the surface. One process of transferring high quality hydrophobic iron oxide nanoparticles to aqueous phase is based on exchanging oleic acid coating with amphiphilic molecules, such as poly(acrylic acid) (PAA), polyethylenimine (PEl), or glutathione (GSH). However, such exchange process for transferring high quality hydrophobic nanoparticles to aqueous phase developed for synthetic polymers is not applicable for protein coating as it requires heating in dipolar solvent (usually dimethyl sulfoxide, DMSO). Chen et al. Preparation and control of the formation of single core and clustered nanoparticles for biomedical applications using a versatile amphiphilic diblock copolymer. See Nano Research 2010, 3, 852-862, Balasubramaniam et al. Poly(N-isopropylacrylamide)-Coated Superparamagnetic Iron Oxide Nanoparticles: Relaxometric and Fluorescence Behavior Correlate to Temperature-Dependent Aggregation. See Chemistry of Materials 2011, 23, 3348-3356 and Meldrum et al. Synthesis of Inorganic Nanophase Materials in Supramolecular Protein Cages. See Nature 1991, 349, 684-687. On the other hand, biomimetic magnetic nanoparticles can be formed by mineralizing metal oxides inside the protein cages. However, the biomineralization or co-precipitation typically takes place under mild conditions, yielding magnetic nanoparticles with limited magnetic properties. Valero et al. Magnetic Nanoparticles-Templated Assembly of Protein Subunits: A New Platform for Carbohydrate-Based MRI Nanoprobes. See Journal of the American Chemical Society 2011, 133, 4889-4895 and Fan et al. Magnetoferritin nanoparticles for targeting and visualizing tumor tissues. See Nature Nanotechnology 2012, 7, 459-464.

Casein protein makes up 80% of the composition of milk and contains several related phosphoproteins. In bovine milk, four types of phosphoproteins exist, namely αS1-, αS2-, β-, and κ-CN, with molecular weight 19-25 kilodaltons, and an isoelectric point 4.2-5.8. Naturally, caseins self-assemble into micelles with diameters ranging from 50-500 nm, acting as natural nanovehicles for delivery of calcium, phosphate and other biomolecules. Generally, the casein micelle is believed to be formed based on the open structure and distinct hydrophobic and hydrophilic domains of these proline-rich phosphoproteins.

Sangeetha et al. report a synthesis of casein coated iron oxide particles however this method employs precipitation reaction that results in magnetic particles with limited magnetic properties. See “The interaction, stability and response to an external stimulus of iron oxide nanoparticles-casein nanocomplexes.” Colloids and Surfaces A: Physicochemical and Engineering Aspects 406 (2012) 52-60. (see also WO 2007/116954)

Gupta et al. report the synthesis of lactoferrin and ceruloplasmin coated iron oxide nanoparticles using 1-ethyl-3-(3-dimethylaminopropyl)-carbodiimide (EDCI) coupling methods. Lactoferrin and ceruloplasmin derivatized superparamagnetic iron oxide nanoparticles for targeting cell surface receptors. See Biomaterials 25 (2004) 3029-3040.

US Patent Application 20120230919 reports the synthesis of a MRI contrast agent composed of carboxylated mannan coated iron oxide nanoparticles.

WO 1999/062079 reports a method for synthesizing iron oxide nanoparticles.

There remains a need for improved contrast agents with magnetic properties that allow high resolution MRI imaging as well as drug delivery systems.

References cited herein are not an admission of prior art.

### SUMMARY

In certain embodiments, this disclosure relates to magnetic iron oxide nanoparticles that are coated in a hydrophilic protein that provides improved solubility while retaining magnetic properties necessary for use in vivo. Generally, the hydrophobic protein is a casein. The high solubility in water allows these nanoparticles to be more biocompatible for in vivo applications. In certain embodiments, this disclosure relates to magnetic casein coated nanoparticles that can be utilized as a contrast agent in targeted imaging by MRI, a vehicle for targeted drug delivery, or in vitro cell sorting. This disclosure provides the method for generating casein coated iron oxide nanoparticles that comprise: a) glucose oxidation and polymerization into oligosaccharides under high temperature; b) exchange of oleic acid with oligosaccharides; and c) encapsulation with a casein protein.

In other aspects, this disclosure relates to the use of casein coated iron oxide nanoparticles as a superparamagnetic MRI contrast agent. In certain embodiments, the casein coated iron oxide nanoparticles have a reduced relaxation time after magnetic stimulation when compared to non-casein coated iron oxide particles, thereby allowing higher resolution imaging. In yet other aspects, this disclosure relates to casein coated nanoparticles as a MRI contrast agent that improves contrast and image quality.

In some aspects, this disclosure relates to the conjugation of bio-affinitive ligands to the surface of the casein coated iron oxide nanoparticles. In some aspects of this disclosure, conjugation of with any bio-affinitive ligand can allow for targeted imaging by MRI. In further aspects, this disclosure relates to a MRI contrast agent that can aid in the diagnosis of diseases.

In further embodiments, this disclosure relates to the conjugation of bio-affinitive ligands on the surface of casein coated iron oxide nanoparticles, wherein the bio-affinitive ligands can specifically bind certain cell types, through cell type specific markers. In further embodiments, this disclosure relates to the conjugation of bio-affinitive ligands on the surface of casein coated iron oxide nanoparticles, wherein the bio-affinitive ligands can specifically bind certain cell types comprising, but not limited to: apoptotic cells, mitotic cells, tumor cells, activated immune cells, activated endothelial cells, bacterial cells, or degenerative cells. This disclosure relates to the conjugation of bio-affinitive ligands on the surface of casein coated iron oxide nanoparticles, wherein the bio-affinitive targeting ligands can target specific tissue, internal organs, or abnormal growths.

In certain embodiments, this disclosure relates to casein coated iron oxide nanoparticles that can be linked with at least one therapeutic agent. In other embodiments, this disclosure relates to casein coated iron oxide nanoparticles that can be linked with at least one therapeutic agent wherein, the therapeutic agent can comprise but is not limited to any of the following group: a chemotherapeutic agent, an antibiotic agent, an antifungal agent, an antiparasitic agent or an antiviral agent. In other embodiments, this disclosure relates to casein coated iron oxide nanoparticles that can be linked with at least one therapeutic agent wherein, the therapeutic agent can comprise but is not limited to any of the following group: a chemotherapeutic agent, an antibiotic agent, an antifungal agent, an antiparasitic agent or an antiviral agent wherein, the said casein coated iron oxide can also be conjugated with any bio-affinitive ligand for targeted drug delivery.

In other aspects, this disclosure relates to use of magnetic casein coated iron oxide nanoparticles as research tool. In other aspects, this disclosure relates to magnetic casein coated iron oxide nanoparticles that can be conjugated with any bio-affinitive ligand for targeted cell sorting applications in vitro. In other aspects, this disclosure relates to magnetic casein coated iron oxide nanoparticles that can be linked with any therapeutic agent comprising, a chemotherapeutic agent, an antibiotic agent, an antifungal agent, an antiparasitic agent or an antiviral agent, wherein the said therapeutic agent linked-casein coated iron oxide nanoparticles can be used as a research tool.

In other embodiments, this disclosure relates to a drug or treatment that comprises casein coated iron oxide nanoparticles conjugated with a bio-affinitive ligand that recognizes a specific cell type, wherein the casein coated iron oxide nanoparticles can also have a therapeutic agent comprising but not limited to an agent from the following group a chemotherapeutic agent, an antibiotic agent, an antifungal agent, an antiparasitic agent or an antiviral agent.

In some aspects, this disclosure relates to a kit containing magnetic casein coated iron oxide nanoparticles. In further embodiments, this disclosure relates to a kit containing magnetic casein coated iron oxide nanoparticles conjugated with any bio-affinitive ligand for targeted cell. In other aspects, this disclosure relates to a kit containing magnetic casein coated iron oxide nanoparticles linked with any therapeutic agent comprising an agent from the following group a chemotherapeutic agent, an antibiotic agent, an antifungal agent, an antiparasitic agent or an antiviral agent. In other aspects, this disclosure relates to a kit containing magnetic casein coated iron oxide nanoparticles conjugated with a bio-affinitive ligand wherein, said conjugated casein coated iron oxide nanoparticles are linked with any therapeutic agent comprising an agent from the following group a chemotherapeutic agent, an antibiotic agent, an antifungal agent, an antiparasitic agent or an antiviral agent.

### BRIEF DESCRIPTION OF THE FIGURES

FIG. 1 illustrates the reaction scheme of the exchange-encapsulation process for coating iron oxide nanoparticles with casein.

FIG. 2A shows TEM images of hydrophobic iron oxide nanoparticles before modification.

FIG. 2B shows TEM images of casein coated iron oxide nanoparticles.

FIG. 2C shows negative staining TEM images of casein coated iron oxide nanoparticles showing a protein coating (light colored).

FIG. 2D are photographs of hydrophobic iron oxide nanoparticles dispersed in chloroform (left) and water-soluble casein coated iron oxide nanoparticles (right) dynamic light scattering data showing the change of the hydrodynamic size of iron oxide nanoparticles before and after coated with casein pictures of gel electrophoresis analysis of (i) casein (ii) oligosaccharide coated iron oxide nanoparticles, (iii) casein coated iron oxide nanoparticles (top), and corresponding gel code blue staining (bottom).

FIG. 3 is a graph showing the FT-IR spectra of hydrophobic iron oxide nanoparticles, oligosaccharide coated iron oxide nanoparticles, and casein coated iron oxide nanoparticles.

FIG. 4 is a graph showing the stability of casein coated iron oxide nanoparticles in water, Phosphate buffered saline (PBS) and RPMI1640 culture medium assessed by dynamic light scattering.

FIG. 5A is T₂-weighted spin echo magnetic resonance images of casein coated iron oxide nanoparticles and SHPI5 at different concentrations.

FIG. 5B shows relaxation rates (1/T₂, s⁻¹) as a function of iron concentration (mM) for casein coated iron oxide nanoparticles and SHP15.

FIG. 6A is plots of hydrodynamic size change (sphere) and the surface charge change (triangle) of casein coated iron oxide nanoparticles as a function of pH.

FIG. 6B is transverse relaxivity r₂ of casein coated iron oxide nanoparticles as a function of pH.

FIG. 7 is a graph showing cell viability of RA W264. 7 macrophage cells treated with various concentrations of casein coated iron oxide nanoparticles for 24 hours measured by the MTT assay.

FIG. 8A is the in vivo T₂-weighted magnetic resonance images of a mouse before and post-injection of casein coated iron oxide nanoparticles at a dosage of 2.5 mg Fe/kg per mouse body weight.

FIG. 8B is a graph showing relative magnetic resonance signal in liver (circle), spleen (triangle) and muscle (square).

FIG. 8C is a graph showing blood circulation half-life time of casein coated iron oxide nanoparticles in mice (n=3/time point) by magnetic resonance imaging.

FIG. 9A is images of Prussian blue staining images of MDA-MB-231 (over-expressing EGFR) and MCF7 (low level expressing EGFR) cells after incubated with ScFvEGFR-CNIO at 37° C. for 4 hours, nanoparticles stained present as blue dots.

FIG. 9B is a graph of Prussian blue-stained cells as a percentage of total analyzed by ImageJ. This revealed pronounced cellular uptake of ScFvEGFR-CNIOs by MDA-MB-231 cells.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

It must be noted that, as used in the specification and the appended claims, the singular forms “a,” “an,” and “the” include plural referents unless the context clearly dictates otherwise. Thus, for example, reference to “a support” includes a plurality of supports.

As used herein, “a casein” or “CN” refers to a casein protein, the predominant protein in non-human mammals and human milk, comprising the subgroups αS1, αS2, β and K, or a derivative thereof such as a caseinate.

“Iron oxide nanoparticle”’ refers to sub-microscopic particles whose overall hydrodynamic diameter is less than 1 μm, as measured by dynamic light scattering (DLS), comprising a core selected from one or more of iron hydroxide, iron oxides, iron oxide hydrates, iron mixed oxides or iron.

As used herein, the term “IO” refers to iron oxide nanoparticles which are coated with oleic acid.

As used herein, the term “SIO” refers to iron oxide nanoparticles which were previously coated with oleic acid that were partially exchanged and coated with oligosaccharides.

As used herein, the term “CNIO” refers to iron oxide nanoparticles which were coated with oligosaccharides and are encapsulated with casein protein.

As used herein, the term “MRI” refers to magnetic resonance imaging that performs a scan of a particular region of interest, which includes gradient coils that generate time-varying gradient magnetic fields along the x, y, and z-axes of the examination region. The MRI produces radio frequency signals which excite or otherwise manipulate hydrogen or other magnetic resonant active nuclei in the patient. Energy emitted is detected by a scanner and converted mathematically into an image.

As used herein, the term “DLS” or “Dynamic Light Scattering” refers to an assay performed to determine size resolution of particles in optical transparent aqueous samples some three orders of magnitude below an optical microscope.

As used herein, the tern “FT-IR spectra” refers to Fourier transform infrared spectra a technique which determines the infrared spectrum of absorption, emission, photoconductivity or Raman scattering of a solid, liquid or gas.

### Methods for Casein Coated Iron Oxide Nanoparticle Synthesis

In certain embodiments, this disclosure provides a method for the robust preparation of casein coated iron oxide nanoparticles. In some aspects the casein is not in the form of a micelle. In certain aspects this disclosure relates to the coating of the iron oxide nanoparticles with casein using exchange-encapsulation wherein this method produces water soluble, superparamagnetic nanoparticles.

In some embodiments, a process to form an iron oxide nanoparticle coated in a protein is provided including: providing an iron oxide nanoparticle with one or more oleic acid residues; exposing the iron oxide nanoparticle to glucose under high temperature, yielding a partially oligosaccharide-coated iron oxide nanoparticle; and encapsulating oligosaccharide-coated iron oxide nanoparticle with a protein. In certain embodiments, the protein is a casein.

In certain embodiments, the iron oxide nanoparticle is magnetite or maghemite. In certain embodiments, iron oxide nanoparticles are generated through pyrolysis using laser or other techniques. In some embodiments, the particles have a core size of between about 10 to 20 nm or about 12 to 18 or about 14 to 16 nm.

Conventional exchange process for transferring high quality hydrophobic iron oxide nanoparticles to aqueous phase developed for synthetic polymers is not applicable for protein coating as it requires heating in dipolar solvent, dimethyl sulfoxide. Such a harsh reaction conditions easily denatures the protein and deforms the nanoparticle surface structure. Certain aspects of this disclosure relates to an exchange-encapsulation method, mediated by glucose, which can be oxidized and polymerized into oligosaccharides under high temperature conditions. In certain embodiments, the disclosure therefore provides a process for generating casein-coated iron oxide nanoparticles comprising: providing an oleic-acid containing iron oxide nanoparticle, contacting the oleic acid containing nanoparticle with glucose at elevated temperatures to provide an oligosaccharide-coated nanoparticle; and contacting the oligosaccharide-coated nanoparticle with casein to form a casein-coated nanoparticle. In certain embodiments, the elevated temperature is at least 100° C., or at least 120° C., or at least 150° C., or at least 170° C. In some embodiments, the process further includes contacting the casein-coated nanoparticle with a further agent, such as a therapeutic or diagnostic agent or a cell-targeting moiety. In certain embodiments, the nanoparticle is water soluble.

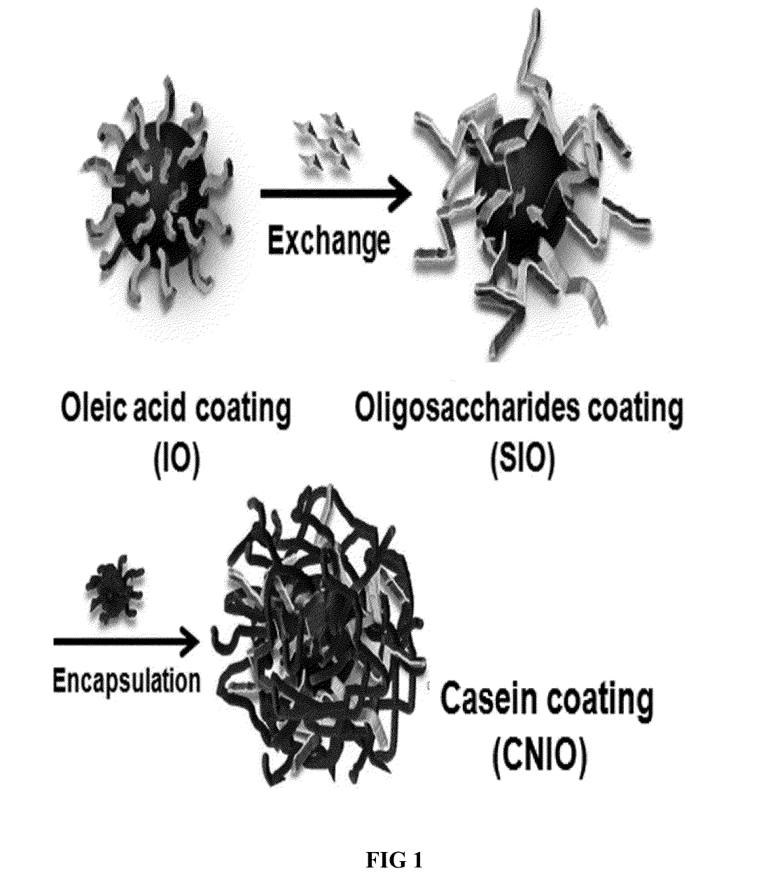

The oligosaccharide molecules partially replace oleic acid and protect iron oxide nanoparticles from oxidation during the exchange procedure (see FIG. 1). The ionic oligosaccharide rendered some hydrophilic moieties and attracted protein molecules onto the surface of iron oxide nanoparticle cores. Resulting casein coated iron oxide nanoparticles may have excellent water solubility and stability. In other embodiments, the increased solubility of casein coated iron oxide nanoparticles renders a biocompatible property that allows these nanoparticles to be used for in vivo applications.

In certain embodiments, this disclosure relates to hydrophobic iron oxide nanoparticles wherein the iron oxide is magnetite (Fe₃O₄) or maghemite (Fe₂O₃). In certain aspects, this disclosure relates to the hydrophobic, oleic acid coated iron oxide nanoparticles that have an initial core size of 15.1±0.6 nm. In other aspects this disclosure relates to iron oxide nanoparticles that are only soluble in nonpolar solvents. In other embodiments this disclosure relates to the exchange of oleic acid from the surface of the hydrophobic iron oxide nanoparticles for oligosaccharides wherein the hydrodynamic size after oligosaccharide exchange increased to 17.6-18.5 nm. In other aspects, this disclosure relates to oligosaccharide coated iron oxide nanoparticles that are subsequently coated with casein, wherein the hydrodynamic size following casein coating increases the iron oxide nanoparticles to 30.5 nm. In other aspects, this disclosure relates to a protein coating surrounding the iron oxide nanoparticles that is 4 nm. In further embodiments, this disclosure relates to the confirmation of casein coating of the iron oxide nanoparticles that result in a change in surface potential from −18.8±4.9 mV to −38.0±3.9 mV.

In some embodiments, the hydrodynamic size of nanoparticles described herein is less than 100 nm or less than 50 nm, or less than about 40 nm, or less than about 30 nm in diameter.

In other aspects, this disclosure relates to casein coated iron oxide nanoparticles that are soluble in aqueous solutions. In other embodiments, milk coated iron oxide nanoparticles can remain stable after months of storage at 4° C., without precipitation. In other embodiments that hydrodynamic size of the casein coated iron oxide nanoparticles can remain unchanged after 48 hours of incubation in phosphate buffered saline and serum containing cell culture medium at physiological conditions, at room temperature.

In some aspects this disclosure relates to casein coated nanoparticles exhibit a significantly higher transverse relaxivity than that of the nanoparticles coated with the conventional polymers, rendering a novel and highly efficient MRI contrast agent. In further aspects this disclosure relates to the conjugation of bio-affinitive ligands, such as single chain fragment from the antibody of epidermal growth factor receptor ligands to casein coated surface of iron oxide nanoparticles that can facilitate targeting of certain cell or tissue types allowing for targeted MRI imaging. In additional aspects, this disclosure relates to the use of conjugated casein coated iron oxide nanoparticles linked with therapeutic agents that can also act as a vehicle for targeted drug delivery.

In certain embodiments, the casein coated iron oxide nanoparticles have a transverse relaxivity r₂ value of at least 150 s⁻¹ mM⁻¹ or at least 175 s⁻¹ mM⁻¹, or at least 200 s⁻¹ mM⁻¹ or at least 250 s⁻¹ mM⁻¹ or between 200-300 s⁻¹ mM⁻¹ or between 250 and 300 s⁻¹ mM⁻¹ or between 250 and 275 s⁻¹ mM⁻¹. In certain embodiments, the nanoparticle has an increased signal-to-noise ratio over a nanoparticle without the casein coating. In some embodiments, the iron oxide nanoparticle coated in a casein, has a transverse relaxivity of at least 200 s⁻¹ mM⁻¹.

In some embodiments, the casein coated iron oxide nanoparticles are 15-40 nm. In some embodiments, the iron oxide nanoparticle is magnetite (Fe₃O₄) or maghemite (Fe₂O₃). In certain embodiments, the casein-coated nanoparticles are further conjugated with any bio-affinitive ligand. In some embodiments, the bioaffinity ligand is for targeted imaging by MRI. In some embodiments, the bio-affinitive ligand is capable of targeting delivery of the particle. In some embodiments, the ligand is capable of specifically binding to certain cell types. In some embodiments, the cells are tumor cells. In certain specific embodiments, the nanoparticle after administration is targeted to liver or spleen cells. In certain embodiments, the nanoparticle is used for diagnosis of a disease. In certain embodiments, the casein-coated nanoparticle includes at least one therapeutic agent. In some embodiments, the therapeutic agent is selected from a chemotherapeutic agent, an antibiotic agent, an antifungal agent, an antiparasitic agent and an antiviral agent. In some embodiments, the casein coated iron oxide nanoparticles linked with a therapeutic agent is also conjugated with any bio-affinitive ligand. In certain embodiments, a composition is provided including a casein coated iron oxide nanoparticle conjugated with a targeting ligand. In certain embodiments, composition also comprises a therapeutic agent.

### Methods of Use

In other aspects, this disclosure provides methods for use of casein coated iron oxide nanoparticles for diagnostic purposes. In certain aspects, this disclosure relates to casein coated iron oxide nanoparticles as a superparamagnetic MRI contrast agent. In other embodiments, this disclosure relates to casein coated iron oxide nanoparticles as a contrast agent with high transverse relaxivity that shortens MRI relaxation time. In some aspects, this disclosure provides casein coated nanoparticles as a MRI contrast agent that improves contrast and image quality. In some aspects, the disclosure provides a method of imaging a host including administering a nanoparticle described herein to the host and imaging the host in an MRI system.

In certain aspects, this disclosure provides an alternative contrast agent to current gadolinium-based MRI contrast agents. In some aspects, this disclosure provides a method of imaging a host with that are or are at risk of acquiring serious illnesses. Particular illnesses include nephrogenic systemic fibrosis or nephrogenic fibrosing dermopathy or renal failure requiring dialysis.

In some aspects, this disclosure relates to the conjugation of bio-affinitive ligands to the surface of the casein coated iron oxide based contrast agent. In certain embodiments, this disclosure relates to the conjugation of bio-affinitive ligands on the surface of casein coated iron oxide nanoparticles, that can facilitate the binding of certain cell types comprising, but not limited to: apoptotic cells, mitotic cells, tumor cells, activated immune cells, activated endothelial cells, bacterial cells, or degenerative cells. In some aspects of this disclosure, conjugation with any bio-affinitive ligand can allow for targeted imaging by MRI allowing for targeted visualization of any internal organs, muscles, connective tissue, or central nervous system. In further aspects, this disclosure relates to a MRI contrast agent that can aid in the diagnosis of diseases comprising but not limited to this group: tumors, cancer metastasis, brain bleeding or swelling, brain aneurysms, stroke, inflammation, arthritis, rheumatoid arthritis, Alzheimer's disease, dementia, coronary artery disease, pericardial disease, heart valve disease, heart muscle disease cardiomyopathy) congenital heart disease, central nervous system demyelination, muscular sclerosis, Crohn's disease, and any other disease that alters the structure of internal organs, connective tissues, or muscles.

In further embodiments, this disclosure relates to the conjugation of bio-affinitive ligands on the surface of casein coated iron oxide nanoparticles, wherein the bio-affinitive ligands can specifically bind certain cell types, through cell type specific markers. In further embodiments, this disclosure relates to the conjugation of bio-affinitive ligands on the surface of casein coated iron oxide nanoparticles, wherein the bio-affinitive ligands can specifically bind certain cell types comprising, but not limited to: apoptotic cells, mitotic cells, tumor cells, activated immune cells, activated endothelial cells, bacterial cells, or degenerative cells. This disclosure relates to the conjugation of bio-affinitive ligands on the surface of casein coated iron oxide nanoparticles, wherein the bio-affinitive ligands can target specific tissue, internal organs, or abnormal growths. In certain embodiments, this disclosure relates to casein coated iron oxide nanoparticles that can be linked with at least one therapeutic agent. In other embodiments, this disclosure relates to casein coated iron oxide nanoparticles that can be linked with at least one therapeutic agent wherein, the therapeutic agent can comprise but is not limited to any of the following group: a chemotherapeutic agent, an antibiotic agent, an antifungal agent, an antiparasitic agent or an antiviral agent. In other embodiments, this disclosure relates to casein coated iron oxide nanoparticles that can be linked with at least one therapeutic agent wherein, the therapeutic agent can comprise but is not limited to any of the following group: a chemotherapeutic agent, an antibiotic agent, an antifungal agent, an antiparasitic agent or an antiviral agent wherein, the said casein coated iron oxide can also be conjugated with any bio-affinitive ligand for targeted drug delivery. In certain instances, the chemotherapy agent is cisplatin.

In further embodiments, this disclosure relates to the conjugation of bio-affinitive ligands on the surface of casein coated iron oxide nanoparticles, wherein the bio-affinitive ligands can specifically bind certain cell types, through cell type specific markers. In further embodiments, this disclosure relates to the conjugation of bio-affinitive ligands on the surface of casein coated iron oxide nanoparticles, wherein the bio-affinitive ligands can specifically bind certain cell types comprising, but not limited to: apoptotic cells, mitotic cells, tumor cells, activated immune cells, activated endothelial cells, bacterial cells, or degenerative cells. This disclosure relates to the conjugation of bio-affinitive ligands on the surface of casein coated iron oxide nanoparticles, wherein the bio-affinitive ligands can target specific tissue, internal organs, or abnormal growths.

In certain embodiments, this disclosure relates to casein coated iron oxide nanoparticles that can be linked with at least one therapeutic agent. In other embodiments, this disclosure relates to casein coated iron oxide nanoparticles that can be linked with at least one therapeutic agent wherein, the therapeutic agent can comprise but is not limited to any of the following group: a chemotherapeutic agent, an antibiotic agent, an antifungal agent, an antiparasitic agent or an antiviral agent. In other embodiments, this disclosure relates to casein coated iron oxide nanoparticles that can be linked with at least one therapeutic agent wherein, the therapeutic agent can comprise but is not limited to any of the following group: a chemotherapeutic agent, an antibiotic agent, an antifungal agent, an antiparasitic agent or an antiviral agent wherein, the said casein coated iron oxide can also be conjugated with any bio-affinitive ligand for targeted drug delivery.

In other aspects, this disclosure relates to use of magnetic casein coated iron oxide nanoparticles as research tool. In other aspects, this disclosure relates to magnetic casein coated iron oxide nanoparticles that can be conjugated with any bio-affinitive ligand for targeted cell sorting applications in vitro. In other aspects, this disclosure relates to magnetic casein coated iron oxide nanoparticles that can be linked with any therapeutic agent comprising, a chemotherapeutic agent, an antibiotic agent, an antifungal agent, an antiparasitic agent or an antiviral agent, wherein the said therapeutic agent linked-casein coated iron oxide nanoparticles can be used as a research tool.

In other embodiments, this disclosure relates to a drug or treatment that comprises casein coated iron oxide nanoparticles conjugated with a bio-affinitive ligand that recognizes a specific cell type, wherein the casein coated iron oxide nanoparticles can also have a therapeutic agent comprising but not limited to an agent from the following group a chemotherapeutic agent, an antibiotic agent, an antifungal agent, an antiparasitic agent or an antiviral agent.

In certain embodiments, the nanoparticles are formulated in a pharmaceutically acceptable carrier. In some embodiments, the pharmaceutically acceptable carrier is suitable for oral delivery. In other embodiments, the carrier is suitable for parenteral or intravenous delivery. The term “pharmaceutically acceptable carrier” refers to a carrier or adjuvant that is administered to a host or patient which, and is sufficiently non-toxic to be delivered in doses sufficient to deliver an effective amount of the compound retains the pharmacological activity of the nanoparticle and any compound linked to it. Examples of suitable carriers include any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

### Kits

The disclosure provides kits for carrying out any of the methods described herein. Kits of the disclosure may comprise casein coated iron oxide nanoparticles, and may further include instructions for carrying out a method described herein. Kits may also comprise casein coated iron oxide nanoparticles that are conjugated with bio-affinitive ligands or linked with a therapeutic agent or both. Kits may also comprise casein coated iron oxide nanoparticles that are linked with a therapeutic agent comprising but not limited to a chemotherapeutic agent, an antibiotic agent, an antifungal agent, an antiparasitic agent or an antiviral agent. Kits may also comprise casein coated iron oxide nanoparticles that are conjugated with bio-affinitive ligands that target certain cell types comprising but not limited to apoptotic cells, mitotic cells, tumor cells, activated immune cells, activated endothelial cells, bacterial cells, or degenerative cells.

In certain embodiment, the disclosure provides kits for synthesizing casein coated iron oxide nanoparticles including at least iron oxide nanoparticles, glucose, and casein protein. In some other aspect, the disclosure provides kits for synthesizing casein coated iron oxide nanoparticles including at least iron oxide nanoparticles, glucose, and casein protein; and instructions for carrying out a method for synthesizing casein coated iron oxide nanoparticles described herein.

In a further aspect, the disclosure provides kits for synthesizing casein coated iron oxide nanoparticles including at least casein coated beads with at least one bio-affinitive ligand. In other aspects, the disclosure provides kits for synthesizing casein coated iron oxide nanoparticles including at least casein coated beads and instructions for carrying out a method for synthesizing casein coated iron oxide nanoparticles described herein.

In further examples, kits of this disclosure consist of at least one reagent specific for casein coated iron oxide nanoparticles. More commonly, kits of the disclosure comprise at least one therapeutic agent or at least one bio-affinitive agent that can be conjugated to the surface of casein coated iron oxide nanoparticles. In some embodiments, kits comprise at least 2, at least 3, at least 4, or at least 5, reagents specific for the casein coated iron oxide nanoparticles.

Kits comprising a single reagent specific for casein coated iron oxide nanoparticles will generally have the reagent enclosed in a container (e.g., a vial, ampoule, or other suitable storage container). Likewise, kits including more than one reagent may also have the reagents in containers (separately or in a mixture).

The instructions relating to the use of the kit for carrying out the disclosure generally describe how the contents of the kit are used to carry out the methods of the disclosure. Instructions may include information as sample requirements (e.g., form, pre-assay processing, and size), steps necessary to synthesizing casein coated iron oxide nanoparticles, steps necessary for conjugated bio-affinitive markers casein coated iron oxide nanoparticles, linking a therapeutic agent to casein coated iron oxide nanoparticles, administering casein coated iron oxide nanoparticles to a subject, and interpretation of results.

Instructions supplied in the kits of the disclosure are typically written instructions on a label or package insert (e.g., a paper sheet included in the kit), but machine-readable instructions (e.g., instructions carried on a magnetic or optical storage disk) are also acceptable. In certain embodiments, machine-readable instructions comprise software for a programmable digital computer for comparing the measured values obtained using the reagents included in the kit.

### Systems for Detection

In certain embodiments, the methods may be implemented by computers, systems, or stored on a computer-readable storage medium as instructions for detecting the casein coated iron oxide nanoparticles.

In some embodiments, the disclosure relates to a system. The system may include a computer having a processor configured to perform the methods of the disclosure. The system may also include or may communicate with a camera or other device that can measures energy or a change in current of an electrode or system configured to subject a sample to testing device.

In some embodiments, the system may include a computer having a processor configured to perform the methods of the disclosure. In certain embodiments, the method contemplates recording measurements and/or diagnosis and/or second line chemotherapy treatment on a computer readable medium as data. In certain embodiments the disclosure, contemplates reporting measurements or diagnosis to the subject, a medical professional, researcher, or a representative thereof. In certain embodiments, the disclosure contemplates transferring recorded data over the internet from a diagnostic or research lab to a computer in a medical or research facility.

In some embodiments, the disclosure relates to a system for measuring and recording the casein coated iron oxide nanoparticles disclosed herein comprising a visual device and computer readable memory.

In some embodiments, the method further comprises outputting quantification results. In some embodiments, the method may further comprise recording the detected changes on a computer-readable medium through a visual device such as a camera or video recorder. In certain embodiments, the disclosure contemplates calculating energy emitted and translating it into a visual image.

In some embodiments, the calculating energy emitted may be outputted from a visual device. In some embodiments, the outputting may include displaying, printing, storing, and/or transmitting the visual image. In some embodiments, the visual image may be transmitted to another system, server and/or storage device for the printing, displaying and/or storing.

The methods of the disclosure are not limited to the steps described herein. The steps may be individually modified or omitted, as well as additional steps may be added.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

In some embodiments, the modules provided within the system may be time synchronized. In further embodiments, the system may be time synchronized with other systems, such as those systems that may be on the medical and/or research facility network.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

The following Examples are provided to illustrate the disclosure, but are not intended to limit the scope of the disclosure in any way.

### EXAMPLES

### Example 1

### Synthesis and Characterization of Casein Coated Iron Oxide Nanoparticles

The hydrophobic iron oxide nanoparticles with a core size of 15.1±0.6 nm, as shown in the TEM image in FIG. 1, are coated with oleic acid and are only soluble in nonpolar solvents such as chloroform and hexane. After being exchanged with oligosaccharides and subsequently coated with casein, the obtained casein coated iron oxides nanoparticles became water-soluble (FIG. 1). The TEM measurement showed that the core size of casein coated iron oxides nanoparticles remained unchanged in size after ligand exchange process (FIG. 1). However, the hydrodynamic size of nanoparticles changed from 17.6 to 18.5 nm after exchanging with oligosaccharides, and further increased to 30.5 nm after the formation of casein coating (FIG. 1), suggesting the expected transformation of surface coating at different stages. Negative staining allows for visualizing a layer of 4 nm thick protein coating around the 10 nanoparticles (FIG. 1). Successfully coating casein molecules to the nanoparticle surface was further confirmed by the observation of change of the surface potential from −18.8±4.9 mV to −38.0±3.9 mV after attachment of casein proteins. Substantial increase in negative surface charge is likely attributed to the presence of a large number of carboxyl groups in casein. Size exclusion gel electrophoresis was used to determine the amount and purity of the obtained casein coated iron oxides nanoparticles. Casein coated iron oxides nanoparticles exhibited a slower moving band (FIG. 1), compared to that of the iron oxide nanoparticles before applying casein coating, indicating the increased size of casein coated iron oxides nanoparticles after protein encapsulation. Gel code blue staining, which stains for the presence of casein, further confirmed the presence of casein coating on the casein coated iron oxides nanoparticle sample with a strong blue band in the location corresponding to the casein coated iron oxides nanoparticles, running behind that of pure casein. While for the iron oxide nanoparticles without casein coating, no such blue band was observed because of the absence of protein.

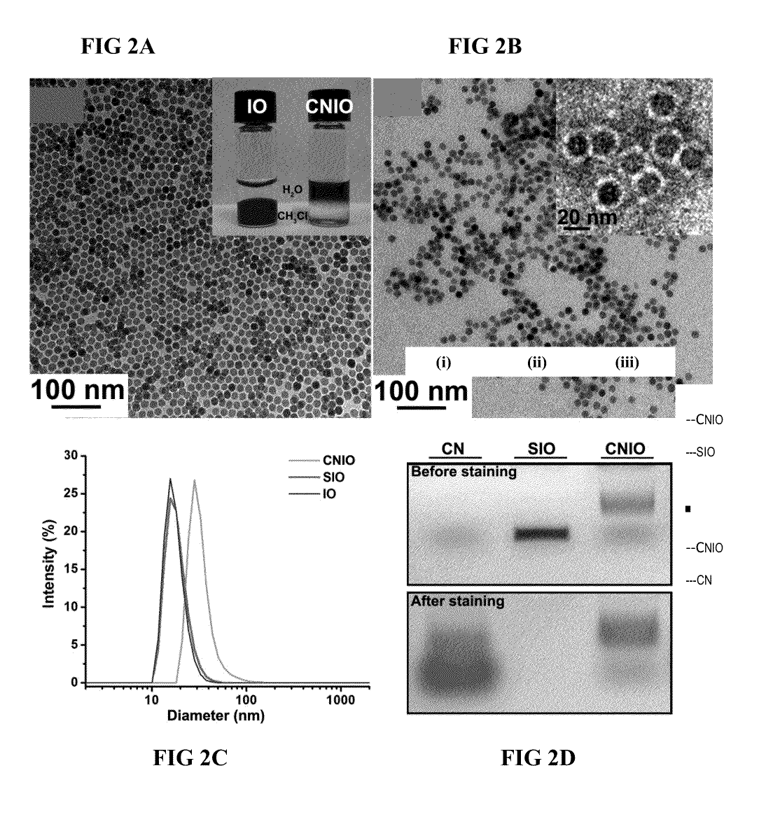

Fourier transform infrared spectra were acquired for further demonstration of the casein coating process (FIG. 2A-D). The presence of casein coating on the nanoparticles was confirmed by the characteristic peaks from amide bands of protein in the IR spectra of casein coated iron oxides nanoparticles. Peaks at 1640 cm⁻¹ and 1530 cm⁻¹ referred to C═O and C—N stretching from amide I and II, and the band at 1300-1200 cm⁻¹ referred to C—N stretching and N—H deformation. Distinguished from casein coated iron oxides nanoparticles, the IR spectra of oleic acid and oligosaccharides coated iron oxide nanoparticles only displayed peaks from C═C stretching and C—H bending. Using the Bradford assay to measure the protein content, we estimated that approximately 50 casein molecules were assembled on each iron oxide nanoparticle.

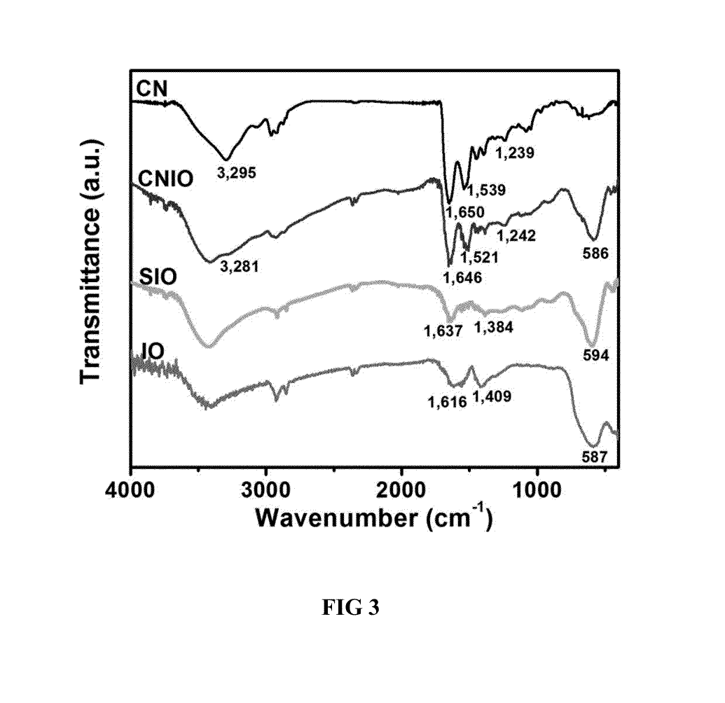

Prepared casein coated iron oxides nanoparticles were easily dispersed in the aqueous solution. Aqueous casein coated iron oxides nanoparticles suspension was stable for months in storage at 4° C. without notable precipitation. Furthermore, the hydrodynamic size of the prepared casein coated iron oxides nanoparticles remained unchanged after 48 hours incubation in the phosphate buffer (PBS, pH 7.4) and serum-contained cell culture medium at the physiological condition at room temperature (FIG. 3).

### Example 2

### MRI Relaxation Properties of Casein Coated Iron Oxides Nanoparticles

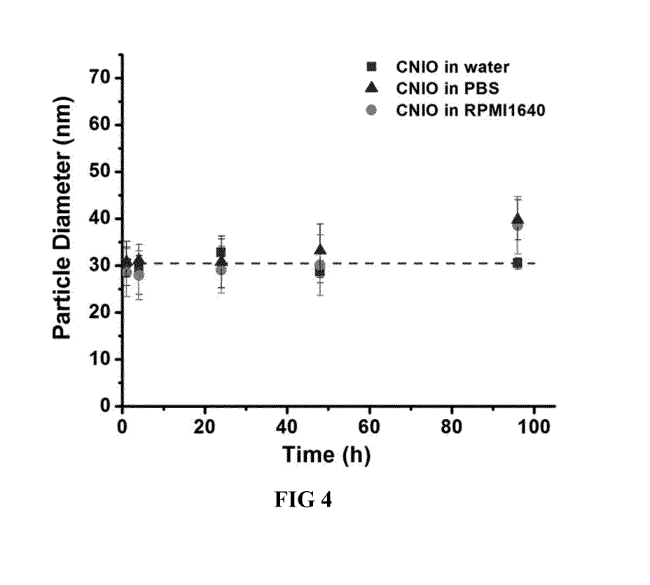

The transverse relaxivity and MRI signal changes of aqueous casein coated iron oxides nanoparticles solutions with different iron concentrations were investigated on a 3 T MRI scanner. For comparison, the same core size (15 nm), water-soluble amphiphilic copolymer coated iron oxide nanoparticles previously developed by us (SHPI5, with carboxyl group, −30˜50 mV) were selected. As shown in FIG. 4, substantially stronger hypointense T₂ weighted spin echo images were seen in the casein coated iron oxides nanoparticles samples compared to those of SHP 15 at the same iron concentrations. Transverse relaxivity r₂ values of iron oxide nanoparticles were calculated from the slope of linear correlation between the relaxation rate 1/T₂ and iron concentrations (FIG. 4). The calculated r₂ is 272.85 s⁻¹ mM⁻¹ for casein coated iron oxides nanoparticles, in comparison to 109.41 s⁻¹ mM⁻¹ for SHP 15 with the same core size. Duan et al. Reexamining the Effects of Particle Size and Surface Chemistry on the Magnetic Properties of Iron Oxide Nanocrystals: New Insights into Spin Disorder and Proton Relaxivity. See Journal of Physical Chemistry C 2008, 112, 8127-8131. With casein coating, casein coated iron oxides nanoparticles exhibited a transverse relaxivity r₂ almost 2.5-fold higher than that of SHP15 coated with amphiphilic copolymer. This is significantly important for MRI application, as increase of the relaxivity leads to high sensitivity for detecting abnormalities or allows for the same imaging quality at a reduced dosage and less toxicity concern. Importantly, observed substantial improvement in transverse relaxivity of casein coated iron oxides nanoparticles suggests that the coating material and its surface properties, e.g., casein structure and properties, play important roles in modulating MRI contrast enhancing effect from the nanoparticle core, likely through changing the interface between the nanoparticle and bulk water medium. Since iron concentration dependent MRI signal decreases due to the dipolar interactions between the magnetic moments of the particles and surrounding water protons, which cause shortening T2 relaxation time and proton dephasing. One possible reason of having higher and efficient MRI contrast from casein coated iron oxides nanoparticles is that the protein coating has a high affinity to water molecules, which promotes the interactions and diffusion of surrounding water molecules around the particles. Moreover, casein molecules have an abundant of hydrate function groups that can increase the exchange between surrounding water molecules and hydrated ones.

### Example 3

### pH Responsive Properties of Casein Coated Iron Oxide Nanoparticles

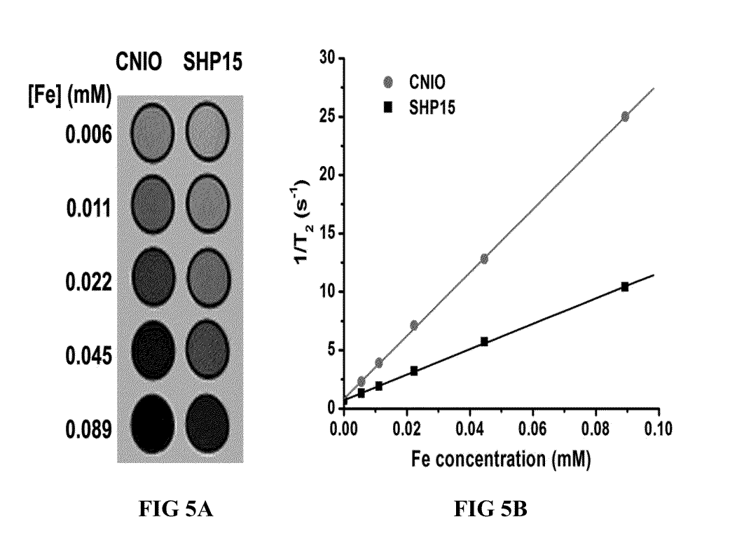

Casein coated iron oxide nanoparticles were found to be highly pH-responsive and exhibit U-shaped dispersibility as a function of solution pH as shown in DLS and zeta potential measurements at different pH conditions. The surface charge of casein coated iron oxide nanoparticles shifted from negative to positive with the decreasing pH value (FIG. 5a). The hydrodynamic size of casein coated iron oxide nanoparticles (˜30 nm) changed slightly in the pH range of 2.0-8.0, except for pH 4.0 which is the isoelectric point (pI) for casein coated iron oxide nanoparticles, when the DLS measured hydrodynamic size sharply increased to 132 nm. The aggregates of casein coated iron oxide nanoparticles at pH 4.0 were dissociated to single dispersed constituent particles, after the pH was adjusted back to 7.0. Though the aggregates formed faster and bigger after the first cycle of pH modulation, this aggregation-dissociation process is reversible up to 5 cycles. TEM images indicated there was no obvious aggregates of casein coated iron oxide nanoparticles existed at pH 7.0 after varying pH in the medium. UV-vis absorption spectra of casein coated iron oxide nanoparticles suspensions at different pH revealed that casein coated iron oxide nanoparticle sample at pH 4.0, showed higher absorption in the spectral range than that at pH 7.0, which also confirmed the aggregation of casein coated iron oxide nanoparticles at pH 4.0. Both single dispersed casein coated iron oxide nanoparticles and aggregated casein coated iron oxide nanoparticles showed the characteristic absorption peak at 280 nm which is attributed to the tryptophan, tyrosine, and cysteine residues in casein.

For the reported casein coated iron oxide nanoparticles, the MRI contrast enhancing effect was fairly constant and stable over the pH range of 4.0-8.0 (FIG. 5b) while casein coated iron oxide nanoparticles aggregate loosely. This unique MRI property is likely to be ascribed to the high affinity for water as well as the porous structure of protein coating, which allows for the nearby water molecules to maintain their molecular motion in the similar microenvironment even after they form the loose aggregates at pi 4.0. The loose structure of casein coated iron oxide nanoparticles aggregates differs from those compact multicore nanoparticle aggregates previously reported in the literatures. Moreover, the distance between the nanoparticles in the casein coated iron oxide nanoparticles aggregates is likely long, so that the interaction between each other is weak. Thus we did not observe the change of overall r₂ relaxivity of casein coated iron oxide nanoparticles aggregates as those compact aggregates reported in the literatures. In fact, the observed constant transverse relaxivity and MRI contrast enhancing effect over the pH range of 4.0-8.0 provided the indirect evidence that the high hydrophilicity and least diffusion restrain for water molecules in the protein layer contribute to much higher r2 values of casein coated iron oxide nanoparticles.

### Example 4

### Cytoxicity of Casein Coated Iron Oxide Nanoparticles

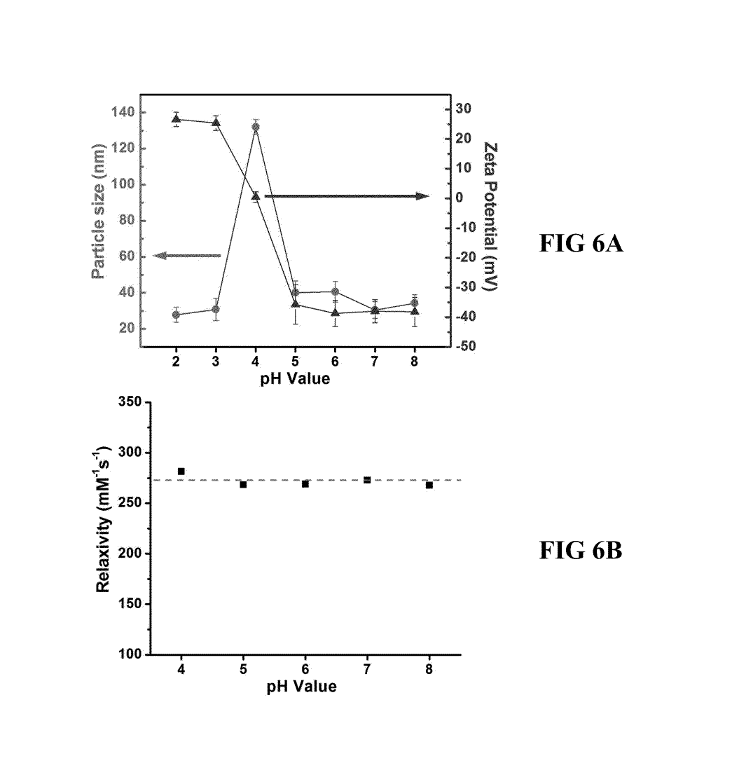

The biocompatibility and cytotoxicity of casein coated iron oxide nanoparticles was evaluated using MTT assay by incubating macrophage RAW264.7 cells with medium containing difference concentrations of casein coated iron oxide nanoparticles, ranging from 5 to 200 μg Fe/mL, for 24 h. The cell viability obtained by the MTT assay was expressed as a fraction of viable cells and normalized to that of cells without co-incubation with casein coated iron oxide nanoparticles as the control. After incubation, cells incubated with casein coated iron oxide nanoparticles survived even at the highest iron concentration at 200 μg Fe/mL, showing viability −90% compared to the control (FIG. 6A-B). These results show that these newly developed casein coated iron oxide nanoparticles are biocompatible and suitable for biomedical applications.

### Example 5

### MRI Contrast Enhancement of Casein Coated Iron Oxide Nanoparticles in Mice

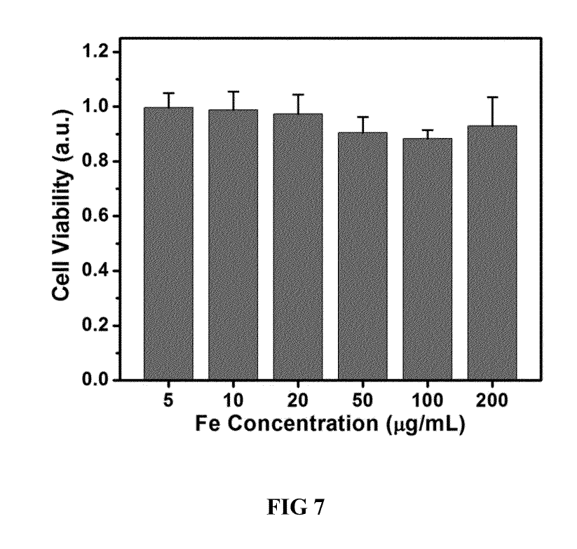

The in vivo MRI contrast enhancing capability of the casein coated iron oxide nanoparticles was tested by intravenously administering it into Balb/c mice at a dosage of 2.5 mg Fe/kg of mouse body weight. As shown in FIG. 7, MRI hypointensities can be readily observed in the liver and spleen in T₂ weighted MR images recorded 20 min after administration of casein coated iron oxide nanoparticles, comparing to the images collected before the injection. The signal-to-noise ratio changed up to ˜76% and ˜52% in liver and spleen, respectively, and began to decrease after 24 hours post injection (FIG. 7). This contrast enhancement indicates the accumulation of casein coated iron oxide nanoparticles in the organs, demonstrating these casein coated iron oxide nanoparticles could be potentially useful for effective T₂-weighted MRI contrast agent.

### Example 6

### Specific Binding of Cancer Cells with EGFR Targeted Casein Coated Iron Oxide Nanoparticles

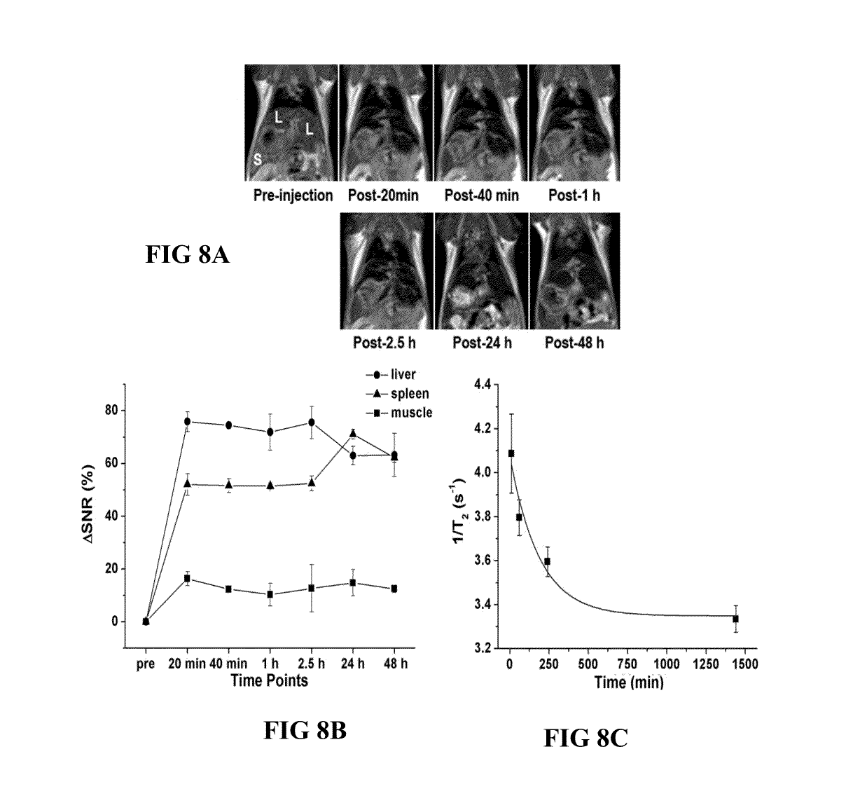

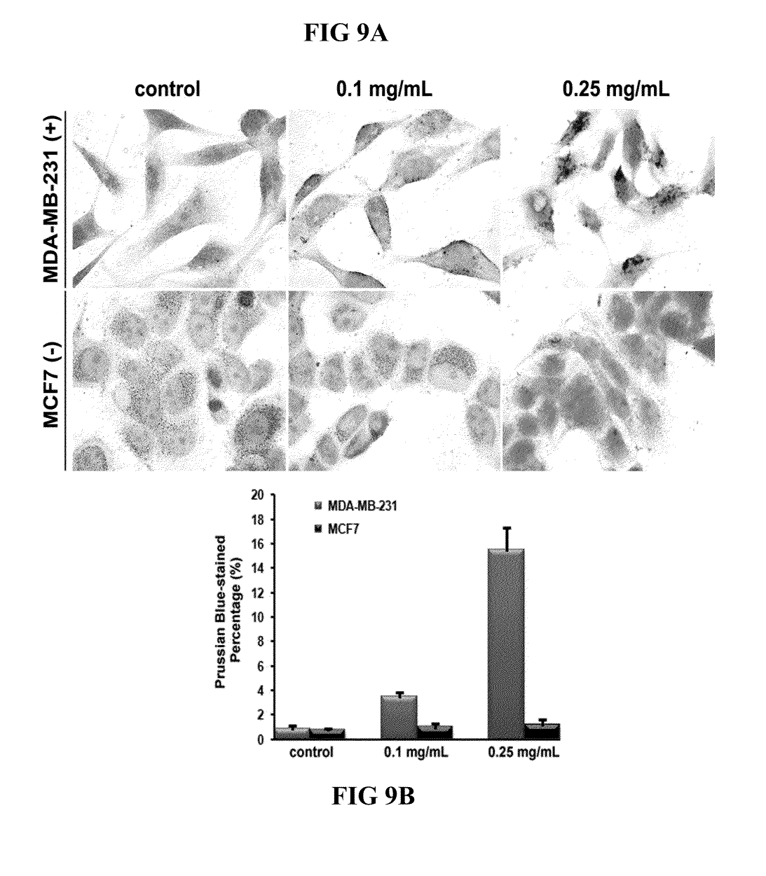

Specific binding of the ScFvEGFR-casein coated iron oxide nanoparticles to MDA-MB-231 human breast cancer cells that have a high level of EGFR was determined by co-incubating cells with ScFvEGFR-casein coated iron oxide nanoparticles followed by Prussian blue staining for iron using a control of breast cancer MCF7 cells with low level expressing EGFR. Marked cellular uptake of ScFvEGFR-casein coated iron oxide nanoparticles by MDA-MB-231 cells in the dose-dependent manner was observed in the Prussian blue staining images (FIG. 8A-C). In comparison, MCF7 cells treated with ScFvEGFR-casein coated iron oxide nanoparticles showed little uptake of EGFR targeted casein coated iron oxide nanoparticles.

We claim:

1. An iron oxide nanoparticle coated in a casein, wherein the particle has a transverse relaxivity of at least 200 s⁻¹ mM⁻¹.

2. An iron oxide nanoparticle coated in a protein formed by a method comprising:
a. providing an iron oxide nanoparticle with one or more oleic acid residues;
b. exposing the iron oxide nanoparticle to glucose under high temperature, yielding a partially oligosaccharide-coated iron oxide nanoparticle; and
c. encapsulating oligosaccharide-coated iron oxide nanoparticle with a protein.

3. A method for synthesizing protein coated iron oxide nanoparticles that comprises:
a. providing an iron oxide nanoparticle with one or more oleic acid residues;
b. exposing the iron oxide nanoparticle to glucose under high temperature, yielding a partially oligosaccharide-coated iron oxide nanoparticle; and
c. encapsulating oligosaccharide-coated iron oxide nanoparticle with a casein.

4. The method of claim 3 wherein the iron oxide nanoparticle is encapsulated in casein protein.

5. The method of claim 4, wherein the casein coated iron oxide nanoparticles are 15-40 nm.

6. The method of claim 3, wherein the iron oxide nanoparticle is magnetite (Fe₃O₄) or maghemite (Fe₂O₃).

7. The method of claim 3 wherein, the casein coated iron oxide nanoparticles are a superparamagnetic MRI contrast agent.

8. The method of claim 7 wherein, the casein coated iron oxide nanoparticles that are a MRI contrast agent shortens relaxation time.

9. The method of claim 4, wherein the casein coated iron oxide nanoparticles is further conjugated with any bio-affinitive ligand.

10. The method of claim 7, wherein the casein coated iron oxide nanoparticles is conjugated with any bio-affinitive ligands for targeted imaging by MRI.

11. The method of claim 7, wherein bio-affinitive ligand is capable of specifically bind to a certain cell types.

12. The method of claim 7, wherein the MRI contrast agent can aid in the diagnosis of disease.

13. The method of claim 3, further comprising linking the casein coated iron oxide nanoparticles to at least one therapeutic agent.

14. The method of claim 13, wherein, the therapeutic agent is a chemotherapeutic agent, an antibiotic agent, an antifungal agent, an antiparasitic agent or an antiviral agent.

15. The method of claim 13, wherein the casein coated iron oxide nanoparticles linked with a therapeutic agent is also conjugated with any bio-affinitive ligand for targeted drug delivery.

16. A composition that comprises a casein coated iron oxide nanoparticle conjugated with a targeting ligand.

17. The composition of claim 16 wherein the casein coated iron oxide nanoparticles also comprises a therapeutic agent selected from the group consisting of a chemotherapeutic agent, an antibiotic agent, an antifungal agent, an antiparasitic agent and an antiviral agent.

18. A kit comprising a magnetic casein coated iron oxide nanoparticle conjugated with at least one targeting ligand.

19. A kit comprising a magnetic casein coated iron oxide nanoparticle linked with at least one therapeutic agent selected from the group consisting of a chemotherapeutic agent, an antibiotic agent, an antifungal agent, an antiparasitic agent and an antiviral agent.
